(19) United States
(12) Patent Application Publication
(10) Pub. No.: US 2011/0305649 A1
(43) Pub. Date: Dec. 15, 2011

(54) High Humectant High Internal Phase Emulsion

(75) Inventors: Anjing Lou, Seymour, CT (US); Qiang Qiu, Trumbull, CT (US); Daniel Alfred Tempesta, Shelton, CT (US)

(73) Assignee: CONOPCO, INC., D/B/A UNILEVER, Englewood Cliffs, NJ (US)

(21) Appl. No.: 12/814,855

(22) Filed: Jun. 14, 2010

(51) Int. Cl. A61K 8/04; A61K 8/34; A61Q 19/00; A61K 8/36; A61K 8/40; A61Q 17/04; A61K 8/60; A61K 8/365

(52) U.S. Cl. 424/59; 514/23; 514/731; 514/557; 514/560; 514/588

(57) ABSTRACT

A high humectant high internal phase emulsion is described. The emulsion has a high content of humectant and less than about 70% by weight water in the internal phase. The emulsion yields excellent moisturizing results when applied and is not sticky during or after application.

### FIELD OF THE INVENTION

The present invention is directed to a high humectant high internal phase emulsion. More particularly, the invention is directed to a high internal phase emulsion, wherein the internal phase occupies at least about 70% by volume of the emulsion and is less than about 70% by weight water based on total weight of the internal phase. The high internal phase emulsion is an excellent base or carrier for actives that are topically applied. Such an emulsion is, unexpectedly, easy to apply, an excellent moisturizing composition and free of unattractive sticky characteristics during and after application.

### BACKGROUND OF THE INVENTION

Water-in-oil emulsions are dispersions of discontinuous or discrete water particles commonly referred to as the internal water phase in a continuous or external oil phase. Certain emulsions can contain as much as or more than 70 volume percent internal phase. Such emulsions are referred to as high internal phase emulsions (HIPEs). The volume fraction of the internal water phase in these types of emulsions can be as high as 90% volume. Often, the internal water phase of some HIPEs can exceed 95% volume or more.

Notwithstanding the fact that use of high internal (water) phase emulsions is known, the formation of stable HIPEs can be difficult. Moreover, many desirable sensory benefits of interest to consumers cannot be obtained with high water content emulsions, especially when the emulsions are topically applied.

It is of increasing interests to develop a high internal phase emulsion that is stable and that is suitable to deliver excellent sensory benefits in the absence of sticky characteristics. This invention, therefore, is directed to a high internal phase emulsion wherein the internal phase occupies at least about 70% by volume of the emulsion and is less than about 70% by weight water. The high internal phase emulsion is an excellent base or carrier for actives that are topically applied. Such an emulsion is, unexpectedly, easy to apply, an excellent moisturizer and free of unattractive sticky characteristics during and after application.

### Additional Information

Efforts have been disclosed for making emulsions. In U.S. Pat. Nos. 6,147,131 and 6,303,834, high internal phase emulsions and foams made therefrom are disclosed.

Other efforts have been disclosed for making emulsions. In U.S. Pat. No. 4,606,913 high internal phase emulsions are described.

Still other efforts have been disclosed for making high internal phase emulsions. In U.S. Patent Application Publication Nos. 2008/0311058 and 2009/0247445, high internal phase emulsions are described.

None of the additional information above describes a HIPE having an internal phase that occupies at least about 70% by volume of the emulsion whereby the total weight of the internal phase is less than about 70% by weight water.

### SUMMARY OF THE INVENTION

In a first aspect, the present invention is directed to an emulsion, the emulsion comprising:
a) an internal phase making up at least about 70% by volume of the emulsion; and
b) an external phase
wherein the internal phase comprises water and a humectant with water making up less than about 70% by weight of the total weight of the internal phase.

In a second aspect, the present invention is directed to a care composition comprising the high humectant high internal phase emulsion of the first aspect of this invention.

In a third aspect, the present invention is directed to a method for treating skin with the aforementioned high humectant HIPE and/or care composition of the previous aspects of this invention.

All other aspects of the present invention will more readily become apparent upon considering the detailed description and examples which follow.

Care composition, as used herein, is meant to include end use cosmetic compositions comprising the high humectant HIPE of this invention and suitable for use by humans to enhance a skin characteristic. High humectant HIPE, as used herein, can be classified as a care composition and especially one suitable to moisturize. Such compositions are preferably classified as leave-on and are meant to include end use compositions that may be, for example, a hair care composition like a conditioner or tonic; lipstick; a leave-on skin enhancer, or a color cosmetic. The preferred end form of HIPE and care composition of this invention is, however, a leave-on composition that displays excellent sensory characteristics in the absence of sticky characteristics generally associated with conventional compositions having high levels of humectant like glycerin (i.e., ≧20% by total weight humectant based on total weight of the conventional composition). The care composition typically will comprise an active (that preferably does more than moisturize) like a sunscreen or a skin lightening agent. Treating is meant to include moisturizing.

Skin, as used herein, is meant to include skin on the face, neck, chest, back, arms, hands, legs and scalp. Excellent sensory characteristics means yielding a comfortable sensation that results in moisturization in the absence of stickiness. Glycerin is meant to mean the same as glycerine and glycerol. High humectant means a HIPE having at least about 30% by weight humectant based on total weight of the internal phase, and often, from about 5 to less than about 60%, and preferably, from about 10 to about 50%, and most preferably, from about 12 to about 45% by weight water based on total weight of the internal phase and including all ranges subsumed therein. High humectant HIPE and HIPE of this invention, as used herein, are meant to mean the same. Steric stabilizer, as used herein, means an ingredient like a polymer (including elastomer) that prevents coalescence of water thereby stabilizing the HIPE. Depletion stabilizer is meant to mean an agent that stabilizes a composition by surrounding water droplets. Free of unattractive sticky characteristics means having a lift force of less than about 0.75 N, and preferably, less than about 0.5N, and most preferably, from about 0.25 to about 0.45 N, including all ranges subsumed therein and as determined by the method described in Example 1.

Unless explicitly stated otherwise, all ranges described herein are meant to include all ranges subsumed therein. The term comprises is meant to include the terms consisting essentially of and consisting of:

### DETAILED DESCRIPTION OF THE PREFERRED EMBODIMENT

The only limitation with respect to the humectant that may be used in this invention is that the same is suitable for use in a topical composition. Illustrative yet non-limiting examples of the types of humectants that may be used in this invention include glycerin; sorbitol; alkylene glycols like propylene glycol, dipropylene glycol, polypropylene glycol, polyethylene glycol, hexylene glycol, 1,3-butylene glycol; C₁₋₃ alkoxylated glucose; hexanetriol; alkoxylated glycerin (like ethoxylated glycerin) or a mixture thereof. In a preferred embodiment, the humectant used is at least about 50% by weight glycerin, based on total weight of humectant. In a most preferred embodiment, the humectant employed consists essentially of glycerin. In a most especially preferred embodiment, the humectant employed in the internal phase of the high humectant HIPE of this invention consists of glycerin.

Regarding the humectant used in the internal phase of the HIPE of this invention, the same typically makes up at least about 30% by weight of the internal phase. In a preferred embodiment, humectant makes up from about 40 to about 95%, and most preferably, from about 55 to about 88% by weight of the internal phase, based on total weight of internal phase and including all ranges subsumed therein. In an especially preferred embodiment, total amount of humectant in the HIPE of this invention is typically from about 25 to about 75%, and preferably, from about 35 to about 70%, and most preferably, from about 40 to 60% by weight, based on total weight of the high humectant HIPE and including all ranges subsumed therein. The HIPE of this invention often comprises from about 3 to about 55%, and preferably, from about 5 to about 45%, and preferably, from about 10 to less than about 40% by weight water, based on total weight of the HIPE of this invention.

Emulsifier that may be used to make the HIPE in this invention often has an HLB of less than about 9, preferably less than about 7, and most preferably, less than about 6. Illustrative examples of the type of emulsifier that may be used in this invention include those generally classified as polyether modified silicone surfactants like PEG/PPG-20/22 butyl ether dimethicone, PEG-3 dimethicone, PEG-9 methyl ether dimethicone, PEG-10 dimethicone, mixtures thereof or the like. The emulsifiers are made available from suppliers like Shin-Etsu and sold under the names KF-6012, KF-6015, KF-6016, and KF-6017, respectively. Another emulsifier suitable for use is DC5225C made commercially available by Dow Corning. In an often preferred embodiment, the emulsifier used in this invention comprises PEG/PPG-18/18 dimethicone, DC5225C or a mixture thereof.

Typically, emulsifier makes up from about 0.5 to about 12%, and preferably, from about 0.8 to about 10%, and most preferably, from about 1 to about 5% by weight of the HIPE of this invention, based on total weight of HIPE and including all ranges subsumed therein.

Steric stabilizer may be used in the external phase of the HIPE of this invention to prevent coalescence of water and to stabilize the high humectant HIPE of this invention. Such a steric stabilizer is preferably an elastomer, and often a cross-linked elastomer (such as a polyether and/or polyglycerine cross-linked silicone elastomer) where the cross-linking group preferably has a chain length from about 8 to about 26 carbon atoms. Moreover, the steric stabilizer is one which preferably has a refractive index of greater than about 1.4 at 25° C.

Often preferred steric stabilizers suitable for use in this invention are Dimethicone/PEG-10/15 Crosspolymer in Dimethicone (KSG-210 or KSG-240), Dimethicone Polyglycerin-3 Crosspolymer in Dimethicone (KSG-710), mixtures thereof or the like. The steric stabilizers are made commercially available, and especially, from suppliers like Shin-Etsu.

Typically, the amount of steric stabilizer (i.e., including carrier they are commercially provided with) employed is from about 0.1 to about 25%, and preferably, from about 0.5 to about 15%, and most preferably, from about 1 to 8%, based on total weight of the high humectant HIPE and including all ranges subsumed therein. In a preferred embodiment, the steric stabilizer used in this invention is KSG-210 or a derivative or mimic thereof.

The high humectant HIPE of the present invention may, within the external phase, further comprise a depletion stabilizer which often is an alkyl modified cross-linked silicone elastomer (such as a polyether and/or polyglycerine cross-linked silicone elastomer) where the cross-linking group preferably has a chain length from about 8 to about 26 carbon atoms.

Illustrative examples of the types of depletion stabilizer suitable for use in this invention include PEG-15/Lauryl Dimethicone Crosspolymer in Mineral Oil (KSG-310), PEG-15/Lauryl Dimethicone Crosspolymer and Isododecane (KSG-320), PEG-15/Lauryl Dimethicone Crosspolymer in Triethylhexanoin (KSG-330), PEG-10/Lauryl Dimethicone Crosspolymer and PEG 15/Lauryl Dimethicone Crosspolymer in Squalane (KSG-340), Lauryl/Dimethicone/Polyglycerine-3 Crosspolymer in Triethylhexanoin (KSG-830), Lauryl Dimethicone/Polyglycerine-3 Crosspolymer in Squalene (KSG-840), mixtures thereof or the like.

When used, the amount of depletion stabilizer (including carrier they are commercially provided with) employed is typically from about 0.25 to about 20%, and preferably, from about 0.5 to about 15%, and most preferably, from about 2 to about 5% by weight, based on total weight of the high humectant HIPE and including all ranges subsumed therein.

In a preferred embodiment, the weight ratio of depletion stabilizer to stearic stabilizer in the HIPE of this invention is from about 1:3 to about 3:1, and most preferably, from about 1:2 to about 2:1. In a most especially preferred embodiment, the amount of steric stabilizer used is from about 1 to about 1.7, and preferably, from about 1 to about 1.5 times the amount by weight of depletion stabilizer used in the HIPE of this invention, including all ranges subsumed therein.

Oil for use in the external phase suitable to generate the HIPE of this invention is limited only to the extent that the same can be used in a composition that may be topically applied. The oil used in the HIPE is preferably silicon-based, and particularly, one classified as dimethicone (DMF-A6cs), a cyclodimethicone such as a D4, D5, or D6 or a mixture thereof whereby such oils are commercially available from suppliers like Shin-Etsu. Other preferred oils suitable for use include dimethicone-based oils having a viscosity from about 3 cps to about 100 cps at ambient temperature and as determined on a Ubbelohde Viscometer. Such oils may be used alone or in combination with other oils suitable for use in topical compositions, like mineral oil and/or paraffin oil.

The oil within the high humectant HIPE of this invention typically makes up from about 0.5 to about 25%, and preferably, from about 5 to about 20%, and most preferably, from about 10 to about 18% by weight of the HIPE, based on total weight of the HIPE of this invention and including all ranges subsumed therein.

In an especially preferred embodiment, less than about 60%, and preferably, less than about 50%, and most preferably, from about 2 to about 35% by weight of the total oil in the HIPE of this invention is provided as carrier with elastomer.

Non-emulsifying elastomer that may be used in the high humectant HIPE of this invention is one which is suitable for use in a composition that may be applied topically. Illustrative yet non-limiting examples of the types of non-emulsifying elastomers that may be used in this invention include those that have an average number (Mn) molecular weight in excess of 2,000, preferably, in excess of 5,000, and most preferably, in the range from about 10,000 to about 20 million, including all ranges subsumed therein. The term “non-emulsifying” defines a siloxane from which polyoxyalkylene units are absent. Often, the elastomers are formed from a divinyl compound which has at least two free vinyl groups, reacting with Si—H linkages of a polysiloxane backbone. Such elastomer compositions are commercially available under the proposed CTFA name of Cyclomethicone and Vinyl Dimethicone Methicone Cross Polymer, delivered as 20-35% elastomer in a cyclomethicone carrier. A related elastomer composition under the CTFA name of Crosslinked Stearyl Methyl Dimethyl Siloxane Copolymer is available as Gransil SR-CYC (25-35% elastomer in a cyclomethicone carrier) from Grant Industries, Inc., Elmwood Park, N.J. The commercial products are typically further processed by subjecting them to a high pressure (approximately 5,000 psi) treatment in a Sonolator with recycling in 10 to 60 passes. Sonolation achieves a resultant fluid with elastomer average particle size ranging from 0.2 to 10 micron, preferably 0.5 to 5 micron. Viscosity is preferred often when ranging between 300 and 20,000 cps at 25° C., as measured by a Brookfield LV Viscometer (size 4 bar, 60 rpm, 15 sec). In an especially preferred embodiment, a most desired non-emulsifying elastomer is a cyclomethicone/dimethicone cross-polymer made commercially available by suppliers like Dow Chemical under the name DC9045, and Shin-Etsu under the name KSG-15 elastomer (with about 5-12% by weight cross-linked polymer in a cyclomethicone carrier).

Typically, the amount of non-emulsifying elastomer (including carrier), used in the HIPE of this invention is from about 1.5 to about 18%, and preferably, from about 2 to about 10%, and most preferably, from about 3 to about 8% by weight, based on total weight of the HIPE and including all ranges subsumed therein.

Care composition comprising the high humectant HIPE of this invention typically further comprises actives or skin benefit agents suitable for addition to the HIPE. Such actives include self-tanning compounds like dihydroxyacetone (DHA), vitamins like niacinamide, vitamin C and its water soluble derivatives, ammonium salts such as those classified as hydroxypropyltri(C₁-C₃ alkyl)ammonium salts, substituted ureas, water soluble resorcinols (including those esterified with, for example, ferulic acid, vanillic acid or the like), moisturizers like sugar derivatives, natural extracts, mixtures thereof or the like.

Illustrative sugar derivatives that may be used include alkylated versions of glucose, sucrose, galactose, xylose, ribose, fructose or mannose, or the like or a mixture thereof. The often preferred sugar derivative is methylglucose. The natural extracts that may be used include, for example, extract of pea, kudzu, yarrow, cucumber, comfrey, chamomile, or a mixture thereof.

The ammonium salts which may be used can be obtained from a variety of synthetic procedures, most particularly by hydrolysis of chlorohydroxypropyl tri(C₁-C₃ alkyl)ammonium salts.

Ordinarily the C₁-C₃ alkyl constituent on the quaternized ammonium group used will be methyl, ethyl, n-propyl, isopropyl or hydroxethyl and mixtures thereof. Particularly preferred is a trimethyl ammonium group known through INCI nomenclature as a “trimonium” group. Any anion can be used in the quat salt. The anion may be organic or inorganic with proviso that the material is cosmetically acceptable. Typical inorganic anions are halides, sulfates, phosphates, nitrates and borates. Most preferred are the halides, especially chloride. Organic anionic counter ions include methosulfate, tolyoyl sulfate, acetate, citrate, tartrate, lactate, gluconate, and benzenesulfonate. A most preferred species of ammonium salt is 1,2-dihydroxypropyltrimonium chloride group.

Illustrative examples of the types of substituted ureas that may be used in this invention include hydroxymethyl urea, hydroxyethyl urea, hydroxypropyl urea; bis(hydroxymethyl) urea; bis(hydroxyethyl) urea; bis(hydroxypropyl) urea; N,N′-di-hydroxymethyl urea; N,N′-di-hydroxyethyl urea; N,N′-di-hydroxypropyl urea; N,N,N′-tri-hydroxyethyl urea; tetra(hydroxymethyl) urea; tetra(hydroxyethyl) urea; tetra(hydroxypropyl) urea; N-methyl-N′-hydroxyethyl urea; N-ethyl-N′-hydroxyethyl urea; N-hydroxypropyl-N′-hydroxyethyl urea and N,N′-dimethyl-N-hydroxyethyl urea. Where the term hydroxypropyl appears, the meaning is generic for either 3-hydroxy-n-propyl, 2-hydroxy-n-propyl, 3-hydroxy-1-propyl or 2-hydroxy-1-propyl radicals. Most preferred is hydroxyethyl urea. The latter is available as a 50% aqueous liquid from the National Starch & Chemical Division of ICI under the trademark Hydrovance.

Other actives suitable for use with the high humectant HIPE of this invention include alpha- and/or beta-hydroxycarboxylic acids, as well as antioxidants. When hydroxycarboxylic acids are employed, they preferably include α-hydroxyethanoic acid, α-hydroxypropanoic acid, α-hydroxyhexanoic acid, α-hydroxyoctanoic acid, α-hydroxydecanoic acid, α-hydroxydodecanoic acid, α-hydroxytetradecanoic acid, α-hydroxyhexadecanoic acid, γ-hydroxyoctadecanoic acid, α-hydroxyeicosanoic acid, α-hydroxydocosanoic acid, α-hydroxyhexacosanoic acid, α-hydroxyoctacosanoic acid, salts thereof, mixtures thereof or the like.

Antioxidants suitable for use include diadzein, genistein, gallic acid, epicatechin, epigallacatechin, epicatechin-3-gallate, epigallocatechin-3-gallate, mixtures thereof or the like.

Still other illustrative actives suitable for use with the HIPE of this invention include resorcinols, retinoids, including retinoic acid, retinal and retinyl esters as well as conjugated linoleic acid (CLA) and/or petroselinic acid, including derivatives thereof.

CLA isomers of the greatest interest in the present invention are cis 9, trans 11-linoleic acid and trans 10, c is 12-linoleic acid. Hereinafter the term “9,11-linoleic” or “10,12-linoleic” shall mean preferentially these two main isomers, but will include lesser amounts of the remaining isomers, particularly when obtained or derived from a natural source.

In accordance with the present invention, 9,11-linoleic acid and 10,12-linoleic acid may be formulated into the HIPE of this invention either as the free acid, as individual chemical derivatives, or as combinations of the free acid and derivative.

By “c9, t11, and 10, c12 isomer enriched CLA” is meant that at least 30% by weight of the total CLA (and/or CLA moieties) that may be present in the HIPE is in the form of the cis 9, trans 11 and trans 10, c is 12 isomers. Preferably, and when used, at least 40%, most preferably at least 50%, by weight of the total CLA and/or CLA moieties present in the HIPE, is in the form of the aforementioned isomers.

Commercially, CLA is available as Clarinol® A-80 and A-95 from Loders-Croklaan, Channahon, Ill. and Neobee® CLA 80 and 90 from Stepan, North Field, Ill.

Typically, the amount of active or skin benefit agent used with the HIPE of this invention is, collectively, from about 0.5 to less than about 30%, and preferably, from about 1 to about 20%, and most preferably, from about 1.5 to about 10%, based on total weight of the care composition and including all ranges subsumed therein.

Internal phase will typically make up the balance of the high humectant HIPE, and should occupy at least about 70% by volume of HIPE to typically no more than about 95% by volume of the HIPE, including all ranges subsumed therein.

Preservatives may also be incorporated into the HIPE of this invention to protect against the growth of potentially harmful microorganisms. While it is in the aqueous phase that microorganisms tend to grow, microorganisms can also reside in the oil phase. As such, preservatives which have solubility in both water and oil are preferably employed in the end use compositions described herein. Suitable traditional preservatives are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are methyl paraben, propyl paraben, imidazolidinyl urea, sodium dehydroxyacetate and benzyl alcohol. The preservatives should be selected having regard for the use of the HIPE and possible incompatibilities between the preservatives and other ingredients in the emulsion. Preservatives are preferably employed in amounts ranging from 0.01% to 2% by weight of the HIPE of this invention.

Conventional thickeners may optionally be used with the HIPE of this invention. Illustrative examples include those commercially sold under the names Aristoflex® AVC (acryloyl dimethyltaurate/vinyl pyrrolidone copolymer), Sepigel®305 (polyacrylaamide/C₁₃₋₁₄ isoparrafin/laureth-7), Simulgel®EG (sodium acrylate/sodium acryloyldimethyl taurate copolymer/isohexadecane/polysorbate 80), Carbopol 934 (crosslinked polyacrylate), Stabylen®30 (acrylates/vinyl isodecanoate crosspolymer) mixtures thereof or the like. The preferred thickener is Aristoflex®AVC made commercially available by Clairiant Corporation. Typically, when employed, the thickener makes up from about 0.01 to about 5.0% by weight of the HIPE, including all ranges subsumed therein.

Sunscreens may be used (in any desirable combination) in the HIPE of this invention and they include those materials commonly employed to block ultraviolet light. Illustrative compounds Avobenzene, available as Parsol 1789®, ethylhexyl-p-methoxycinnamate, available as Parsol MCX®, and benzophenone-3, also known as Oxybenzone. Inorganic sunscreen actives may be employed such as microfine titanium dioxides, polyethylene and various other polymers. The exact amount of sunscreen employed in the compositions can vary depending upon the degree of protection desired from the sun's UV radiation.

Even other optional actives may be used with the HIPE of this invention and they include physical scatterers (like TiO₂ and/or ZnO), chelators (like EDTA), microspheres (e.g., polyethylene based spheroids sold under the name CL-2080; ethylene and methacrylate based spheroids sold under the names SPCAT-I2 and DSPCS-I2, respectively, made available by Kobo Industries), anti-inflammatory agents (including the standard steroidal and non-steroidal types), and dispersants (e.g., PEG-100 stearate and/or NaCl).

When cosmetically acceptable carriers are desired for use with the HIPE of this invention (acting as co-carriers with the HIPE), such carriers may be selected from hydrocarbons, fatty acids, fatty alcohols and esters. Petrolatum is the most preferred hydrocarbon type of emollient conditioning agent. Other hydrocarbons that may be employed include mineral oil, polyolefins such as polydecene, and paraffins such as isohexadecane (e.g., Permethyl 99® and Permethyl 1010).

Fatty acids and alcohols suitable for use as carriers often have from 10 to 30 carbon atoms. Illustrative of this category are pelargonic, lauric, myristic, palmitic, steric, isosteric, hydroxysteric, oleic, linoleic, ricinoleic, arachidic, behenic and erucic acids and alcohols.

Oily ester emollients suitable for use as cosmetically acceptable carriers in topical compositions used in the method of this invention can be those selected from one or more of the following classes:
1. Triglyceride esters such as vegetable and animal fats and oils. Examples include castor oil, cocoa butter, safflower oil, cottonseed oil, corn oil, olive oil, cod liver oil, almond oil, avocado oil, palm oil, sesame oil, squalene, Kikui oil and soybean oil.
2. Acetoglyceride esters, such as acetylated monoglycerides.
3. Ethoxylated glycerides, such as ethoxylated glyceryl monostearate.
4. Alkyl esters of fatty acids having 10 to 20 carbon atoms. Methyl, isopropyl, and butyl esters of fatty acids are useful herein. Examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate.
5. Alkenyl esters of fatty acids having 10 to 20 carbon atoms. Examples thereof include oleyl myristate, oleyl stearate, and oleyl oleate.
6. Ether-esters such as fatty acid esters of ethoxylated fatty alcohols.
7. Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol polyfatty esters, ethoxylated glyceryl monostearate, 1,2-butylene glycol monostearate, 1,2-butylene glycol distearate, polyoxyethylene polyol fatty acid ester, sorbitan fatty acid esters, and polyoxyethylene sorbitan fatty acid esters are satisfactory polyhydric alcohol esters.
8. Wax esters such as beeswax, spermaceti, myristyl myristate, stearyl stearate.

Minor adjunct ingredients may also be included such as fragrances, antifoam agents, and colorants, each in their effective amounts to accomplish their respective functions. In a preferred embodiment, the HIPEs of this invention are substantially free (i.e., less than about 1.0% by weight) of non-emulsifying elastomer, and most preferably, free of non-emulsifying elastomer.

A more detailed description of the types of ingredients that may be used in this invention is found in U.S. Patent Publication Nos. 2008/0311058 A1 and 2009/0247445 A1, the disclosures of which are incorporated herein by reference.

When making the high humectant HIPE or care composition of the present invention, the desired ingredients can be mixed in no particular order and usually at temperatures from about ambient to about 65° C. and under atmospheric pressure. In a preferred embodiment, however, water with water soluble active dissolved therein is added to oil, and the high humectant HIPE is made prior to adding cosmetically acceptable carriers (i.e., co-carriers). Typically mixing occurs at about moderate shear.

The high humectant HIPE and care composition prepared therefrom of this invention preferably have an initial viscosity of less than about 50,000 cps, and most preferably, from about 1,000 to about 40,000 cps (taken at a shear rate of one 1/S, ambient temperature with an Ares Rheometer), including all ranges subsumed therein.

The packaging for the compositions of this invention is not limited and often is a bottle, tube, roll-ball applicator, squeeze container or lidded jar.

The following examples are provided to facilitate an understanding of the present invention. The examples are not intended to limit the scope of the claims.

### Example 1

High humectant high internal phase emulsions consistent with this invention were made by mixing the following ingredients:

TABLE I

| Ingredients | Percent by Weight |
|---|---|
| Water | Balance |
| Glycerin | 40-60 |
| DC5225C | 3 |
| KSG340 | 2 |
| KSG210 | 3 |
| DC9045 | 5 |
| DC245 | 9-12 |
| DMF-A6 cs | 1.5 |
| CL2080 | 5 |
| Aristoflex AVC | 0-2 |

Mixing vessels were charged with ingredients. Water, thickener and humectant were mixed in one vessel and all other ingredients were mixed in a separate vessel. The resulting mixtures were combined and mixed to yield homogeneous emulsions. Mixing was performed at 25° C. and under conditions of atmospheric pressure.

The resulting compositions were assessed for viscosity and lift force or normal force. Viscosity and lift force were assessed with an Ares Rheometer from TA Instruments. Temperature was maintained at 25° C. Lift force (Newtons) was determined with initial gap set at 0.5 millimeters with a 2.5 centimeter parallel geometry being applied. All results obtained for the high humectant high internal phase emulsion of this invention were compared to conventional topical compositions having about 40% by weight glycerin and made available by Neutrogena under the Norwegian Hand Cream Brand.

TABLE II

| Samples | | Viscosity | Viscosity |
|---|---|---|---|
| Compositions made | Lift | Pa · s | Pa · s |
| from Table 1 | Force | Shear rate | Shear rate |
| Ingredients | (N) | 0.01 1/S | 1 1/S |
| 40% glycerin | 0.31 | 1690 | 22 |
| 50% glycerin | 0.41 | 2085 | 38 |
| 60% glycerin | 0.43 | 2159 | 39 |
| Norwegian Hand | 1.18 | 4644 | 156 |
| Cream (fragrance) | | | |
| Norwegian Hand | 1.15 | 4439 | 143 |
| Cream (age shield) | | | |

The results indicate that the high humectant HIPE of this invention unexpectedly maintain a low viscosity in comparison to conventional formulations with about equal or less amounts of glycerin. The results also indicate that the high humectant HIPEs of this invention unexpectedly maintain a low lift force in comparison to conventional formulations, and therefore, are, unexpectedly, less sticky than formulations with about equal or less amounts of humectant like glycerin.

### Example 2

High humectant HIPEs, similar to those prepared in Example 1, were applied by skilled panelists (about 2.5 mg composition per cm² skin). All panelists concluded that the HIPEs made according to this invention were easy to apply, were not sticky and resulted in excellent moisturizing characteristics. The panelists also concluded that the HIPEs of this invention performed well (i.e., resulted in skin moisturization) and were less sticky than commercially available formulas having about 40% by weight glycerin formulated therein.

What is claimed is:

1. An emulsion comprising:
(a) an internal phase making up at least about 70% by volume of the emulsion; and
(b) an external phase
wherein the internal phase comprises water and a humectant with water making up less than about 70% by weight of the total weight of the internal phase.

2. The emulsion according to claim 1 wherein the internal phase makes up to 95% by volume of the emulsion.

3. The emulsion according to claim 1 wherein the humectant makes up from about 40 to about 95% by weight of the internal phase.

4. The emulsion according to claim 1 wherein the humectant makes up from about 55 to about 88% by weight of the internal phase.

5. The emulsion according to claim 1 wherein the humectant is glycerin, sorbitol, propylene glycol, dipropylene glycol, polypropylene glycol, polyethylene glycol, hexylene glycol, 1,3-butylene glycol, C₁₋₃ alkoxylated glucose, hexamatriol, alkoxylated glycerin or a mixture thereof.

6. The emulsion according to claim 1 wherein the emulsion further comprises emulsifier, stearic stabilizer, oil, non-emulsifying elastomer, depletion stabilizer or a mixture thereof.

7. The emulsion according to claim 1 wherein the internal phase comprises from about 5 to about 60% by weight water.

8. The emulsion according to claim 1 wherein the internal phase comprises from about 12 to about 45% by weight water.

9. A care composition comprising:
(a) an emulsion comprising:
(i) an internal phase making up at least about 70% by volume of the emulsion; and
(ii) an external phase; and
(iii) active
wherein the internal phase comprises water and a humectant with water making up less than about 70% by weight of the total weight of the internal phase.

10. The care composition according to claim 9 wherein the internal phase makes up to 95% by volume of the emulsion.

11. The care composition according to claim 9 wherein the humectant makes up from about 40 to about 95% by weight of the internal phase.

12. The care composition according to claim 9 wherein the humectant makes up from about 55 to about 88% by weight of the internal phase.

13. The care composition according to claim 9 wherein the humectant is glycerin, sorbitol, propylene glycol, dipropylene glycol, polypropylene glycol, polyethylene glycol, hexylene glycol, 1,3-butylene glycol, C₁₋₃ alkoxylated glucose, hexamatriol, alkoxylated glycerin or a mixture thereof.

14. The care composition according to claim 9 wherein the emulsion further comprises emulsifier, stearic stabilizer, oil, non-emulsifying elastomer, depletion stabilizer or a mixture thereof.

15. The care composition according to claim 9 wherein the humectant consists essentially of glycerin.

16. The care composition according to claim 9 wherein the humectant consists of glycerin.

17. The care composition according to claim 9 wherein the active is a self-tanning compound, vitamin, sugar derivative, ammonium salt, substituted urea, resorcinol, antioxidant, alpha-hydroxycarboxylic acid, beta-hydroxycarboxylic acid, conjugated linoleic acid, petroselinic acid, sunscreen, physical scatterer or a mixture thereof.

18. The care composition according to claim 9 wherein the active is a self-tanning agent and is DHA.

19. The care composition according to claim 9 wherein the active is a vitamin and is niacinamide.

20. A method for treating skin by contacting skin with the emulsion of claim 1 or the care composition of claim 9
